(19) United States
(12) Patent Application Publication
(10) Pub. No.: US 2004/0082588 A1
(43) Pub. Date: Apr. 29, 2004

(54) Methods for treating disorders of calcium homeostasis

(75) Inventors: Ellen W. Evans, Sparta, NJ (US); Wai Nang Choy, Sparta, NJ (US); Elmer J. Mirro, Wantage, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/673,888

(22) Filed: Sep. 29, 2003

Related U.S. Application Data
(60) Provisional application No. 60/414,948, filed on Sep. 30, 2002.

(51) Int. Cl. A61K 31/496; A61K 31/473

(52) U.S. Cl. 514/253.03; 514/290

(57) ABSTRACT

The present invention provides methods for using tricyclic amide compounds, such as

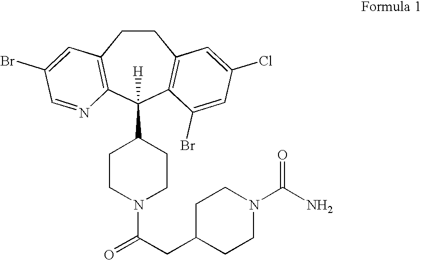

to treat disorders of calcium homeostasis. Such disorders include familial benign hypocalciuric hypercalcemia, neonatal severe primary hyperparathyroidism and renal secondary hyperparathyroidism.

This application claims the benefit of U.S. Provisional Patent Application No. 60/414,948, filed Sep. 30, 2002, which is herein incorporated by reference in its entirety.

### FIELD OF THE INVENTION

The present invention relates to methods for treating calcium homeostatic disorders in a subject.

### BACKGROUND OF THE INVENTION

The complex interplay between calcitropic hormones and their Ca²⁺-translocating target tissues is crucial for maintaining the extracellular/serum calcium concentration in humans within its normal range. These hormones include, inter alia, the parathyroid hormone (PTH); others include calcitonin and 1,25-dihydroxyvitamin D₃. The actions of these hormones regulate renal Ca²⁺ reabsorption, intestinal Ca²⁺ absorption and skeletal Ca²⁺ mobilization. A key element in this process is a G-protein coupled receptor (GPCR) termed the Calcium Ion Sensing Receptor (CaSR). Human CaSR was originally cloned from the parathyroid gland and was subsequently identified in several other tissues including the kidney (Aida, et al., (1995) Biochem. Biophys. Res. Comm. 214:524-529 and Garrett, et al., (1995) J. Biol. Chem. 270:12919-12925). Furthermore, CaSR was identified in several species including X. laevis, rabbits, rats and cows (Brown, et al., (1993) Nature 366:575-580; Riccardi, et al., (1995) Proc. Natl. Acad. Sci. 92:131-135 and Butters, et al., (1997) J. Bone Mineral Res. 12:568-579). CaSR may regulate serum calcium levels by modulating the level of synthesis and secretion of PTH and by modulating the extent of Ca²⁺ reabsorption in the kidneys (Chattopadhyay, (2000) Int. J. Biochem. Cell Bio. 32:789-804; Chattopadhyay, et al., (2000) Cellular Signal. 12:361-366).

An example of a calcium homeostatic disorder is familial hypocalciuric hypercalcemia (FHH), which is sometimes called familial benign hypercalcemia (FBH) or familial benign hypocalciuric hypercalcemia (FBHH). Subjects with FHH suffer from lifelong mild to moderate hypercalcemia. As those with FHH age, they can develop calcium deposits in their cartilage and suffer bouts of acute pancreatitis (Van haeften, et al., (1994) Neth. J. Med. 45(3):110-113; Davies, et al., (1981) Br. Med. J. (Clin. Res. Ed.) 282(6269): 1023-1025).

Neonatal severe hyperparathyroidism (NSHPT) is a condition which can lead to marked bony demineralization, multiple fractures and rib cage malformation. This condition can be fatal if a parathyroidectomy is not carried out within the first few weeks of life (Chattopadhyay, et al., (1996) Endocrin. Rev. 17(4): 289-307). NSHPT may be linked to decreased CaSR activity which, in some cases, may be associated with a CaSR mutation (Chattopadhyay, et al., (1996) Endocrine Rev. 17: 289-307; Heath, et al., (1996) J. Clin. Endocrin. Metab. 81:1312-1317; Pearce, et al., (1995) J. Clin. Invest. 96:2683-2692 and Pollak, et al., (1993) Cell 75: 1297-1303).

Renal secondary hyperparathyroidism is characterized by an excess of serum PTH which is a secondary effect caused by renal failure. Subjects with renal secondary hyperparathyroidism can suffer from disabling skeletal diseases such as renal osteodystrophy (Sherrard, et al., (1993) Kidney Int. 43:436-442; Torres, et al., (1995) Kidney Int. 47: 1434-1442; Moniere-Faugere, et al., (1996) Nephrol. Dial. Transplant. 11: 111-120).

One of the most common causes of hypercalcemia is malignancy (i.e., malignancy associated hypercalcemia (MAH) or humoral hypercalcemia of malignancy (HHM)). It is estimated that 10%-20% of cancer patients suffer from hypercalcemia. The most common cancers that are associated with the development of MAH are squamous cell lung cancer, squamous cell head and neck cancers, breast cancer, multiple myeloma, T-cell lymphomas, renal cell cancer and ovarian cancer. A proximal cause of MAH is believed to be increased bone resorption. Subjects suffering from MAH can suffer from nausea, vomiting, lethargy, confusion and, eventually, death.

Parathyroid hormone has been known since the 1930s to have catabolic effects in bone (e.g., causing resorption of calcium from bone to serum). One medical condition which is characterized by increased bone resorption and increased frequency of bone fractures is osteoporosis. Excess serum parathyroid hormone is likely to be an exacerbating factor in osteoporosis. Thorsen, et al., (1997) (Surgery 122(5):882-887) demonstrated an improvement in bone density in postmenopausal women with hyperparathyroidism after parathyroidectomy.

Subjects suffering from any of the above-mentioned conditions (e.g., FHH, NSHPT, renal secondary hyperparathyroidism, MAH, HHM or osteoporosis) may benefit from a therapy which leads to a decrease in serum Ca²⁺ and/or serum parathyroid hormone levels. The present invention provides, inter alia, methods for treating disorders of calcium homeostasis in a subject, such as those discussed above, by administering a tricyclic amide compound of the invention. Tricyclic amides, such as SCH66336, have been described previously (Liu, et al., (1998) Cancer Res. 58:4974-4956; Njoroge, et al., (1998) J. Med. Chem. 41:1561-1567; U.S. Pat. No. 5,719,148; U.S. Pat. No. 5,874,442; PCT Publication No. WO95/10516) and found to inhibit Farnesyl Protein Transferase (FPT) mediated farnesylation of the Ras protein and, thus, to be useful for treating malignancy.

The use of a farnesylation inhibitor, B-i1086 (Nagasu, et al., (1995) Cancer Res. 55:5310-5314), to treat malignancy-associated hypercalcemia (MAH) has been studied previously (Aklilu, et al., (1997) Cancer Res. 57:4517-4522). Parathyroid Hormone Related Peptide (PTHRP) has been identified as a pathogenic factor in MAH (Moseley, et al., (1987) Proc. Natl. Acad. Sci. USA 84:5048-5052; Stewart, et al., (1987) Biochem. Biophys. Res. Comm. 146:672-678; Stewler, et al., (1987) J. Clin. Invest. 80:1803-1807; Rabbani, et al., (1986) Endocrinology 118:1200-1210; Li, et al., (1994) Cancer Res. 53:2980-2986) and Aklilu et al. found Ras to enhance PTHRP production in rat 3T3 cells. Treatment of mice bearing Ras-3T3 tumors and suffering from hypercalcemia (MAH), with B- 1086, resulted in a normalization of serum calcium and reduction of serum PTHRP levels. B-1086, however, is a peptidomimetic inhibitor which is chemically unrelated to tricyclic amides such as SCH66336. The effect of B-1086 on serum PTH levels was not investigated.

### SUMMARY OF THE INVENTION

The present invention provides a method for treating or preventing disorders of calcium homeostasis (e.g., hypercalcemia) in a subject comprising administering, to the subject, a compound according to the following Formula A:

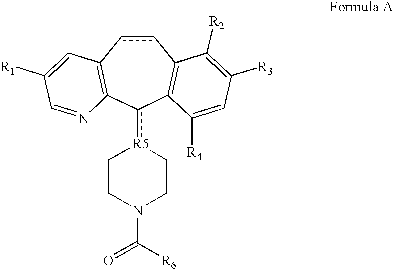

wherein R1 can be hydrogen (H) or halogen (e.g., F, Cl or Br) R2, R3 and R4 are independently selected from H and halogen (e.g., F, Cl or Br) provided that at least one of R2, R3 and R4 is H;

“—” represents an optional bond;

R5 represents C when the optional bond to R5 is present and represents CH or N when the optional bond to R5 is absent;

R6 is selected from

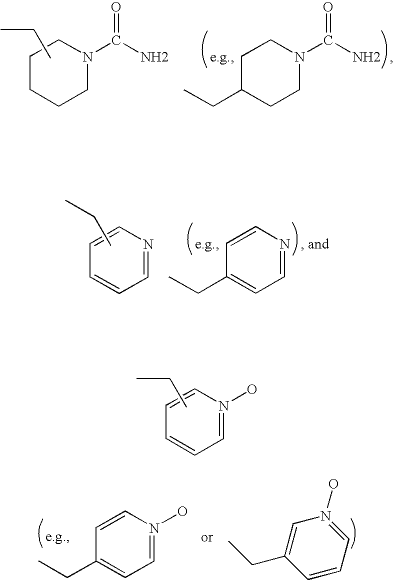

Preferably, the compound is Formula 1 (see infra). Medical conditions which may be treated by the methods of the present invention include familial benign hypocalciuric hypercalcemia, neonatal severe primary hyperparathyroidism and renal secondary hyperparathyroidism.

The present invention also includes methods for agonizing the CaSR, or for decreasing the level of Ca²⁺, PTH or PTHrP in the serum of a subject by administering a tricyclic amide compound, for example, comprising formula A, preferably comprising any of formulas 1-81, more preferably formula 1.

The tricyclic amide compounds of the present invention may be administered in association with a second substance for treating or preventing a calcium homeostatic disorder (e.g., AMG073, NPS467, NPS568, gadolinium, lanthanum, neomycin, Mg²⁺, 1,25-dihydroxyvitamin D, calcitrol, paricalcitrol, doxercalciferol, zoledronic acid, calcitonin, alfacalcidol or oxacalcitriol).

### DETAILED DESCRIPTION OF THE INVENTION

The present invention provides, generally, methods for treating disorders of calcium homeostasis (e.g., hypercalcemia). Specifically, the present invention provides methods for treating calcium homeostatic disorders by administering a tricyclic amide of the invention (e.g., Formula 1) to a subject in need of such treatment. Calcium homeostatic disorders which may be treated by administering the tricyclic amides of the present invention (e.g., Formula 1) include disorders associated with excess serum Ca²⁺ and/or PTH and/or with an excess mobilization of Ca²⁺ from the bones. Such disorders include, but are by no means limited to, familial benign hypocalciuric hypercalcemia, neonatal severe primary hyperparathyroidism, renal secondary hyperparathyroidism, osteoporosis, malignancy-associated hypercalcemia (MAH) and humoral hypercalcemia of malignancy (HHM). Without being bound by a single theory, some calcium homeostatic disorders (e.g., hypercalcemia) may be caused by, for example, an excess of serum PTH, or PTHrP or a lack of expression or activity of the CaSR. Again, without being bound by a single theory, the tricyclic amides of the present invention (e.g., Formula 1) may treat calcium homeostasis disorders (e.g., hypercalcemia) by, for example, agonizing the CaSR (e.g., CaSR in the parathyroid gland or in the kidney) which may, in turn, lead to a decrease in serum PTH or PTHrP. The present invention is not limited by any particular mechanism by which a tricyclic amide of the invention (e.g., Formula 1) may treat or prevent a calcium homeostatic disorder.

The term “Calcium Ion Sensing Receptor”, “Calcium Sensing Receptor” or “CaSR” refers to a receptor which is commonly known in the art. A typical human CaSR amino acid sequence is set forth in SEQ ID NO: 1 as well as under Genbank Accession No. U20759, U20760 and D50855 (Garrett, et al., (1995) J. Biol. Chem. 270(21): 12919-12925; Aida, et al., (1995) Biochem. Biophys. Res. Comm. 214(2):524-529). A typical rat CaSR amino acid sequence is set forth in SEQ ID NO: 2 as well as under Genbank Accession No. U10354 (Riccardi, et al., (1995) Proc. Natl. Acad. Sci. USA 92 (1):131-135). The term includes receptors from any species, preferably mammalian species (e.g., rat, mouse, monkey, rabbit, cow) and most preferably from humans.

The term “parathyroid hormone” or “PTH” is commonly known in the art and refers to the hormone secreted by the parathyroid gland. A typical, mature, human PTH is described by Varicek, et al., (1983) Proc. Natl. Acad. Sci. 80 (8), 2127-2131 and Hendy, et al., (1981) Proc. Natl. Acad. Sci. 78(12): 7365-7369). A typical, mature, rat PTH is described by Schmelzer, et al., (1987) Nucleic Acids Res. 15(16): 6740. The term includes PTH from any species, preferably mammalian species (e.g., dog, cat, pig, rat, mouse, monkey, rabbit, cow) and most preferably from humans.

“PTHrP” refers to hormone, parathyroid hormone related protein, secreted by tumor cells which produces effects similar to those of PTH (see, for example, Suva et al., (1987) Science 237 (4817):893-896).

The term “subject” includes any organism, preferably a mammal (e.g., dog, cat, pig, rat, mouse, monkey, rabbit, cow) and, most preferably, a human.

The term “compound” includes small molecules (e.g., tricyclic amides, polymers, organic molecules, hydrocarbons, inorganic ions and salts), proteins (e.g., antibodies, oligopeptides, polypeptides, hormones and enzymes), saccharides (e.g., monosaccharides, oligosaccharides and polysaccharides) and nucleic acids (e.g., oligonucleotides, polynucleotides, genes, plasmids, DNA and RNA).

The term “e.g. ” means “exempli gratia” or “for example” and, in general, precedes one or more non-limiting examples.

### Tricyclic Amides

Disorders of calcium homeostasis, in a subject, may be treated by administering a tricyclic amide compound to the subject. Preferably, the tricyclic amide compound comprises a formula set forth in U.S. Pat. No. 5,719,148 or in U.S. Pat. No. 5,874,442 which are herein incorporated by reference in their entireties. Preferably, the tricyclic amide compounds of the present invention include the following Formula A:

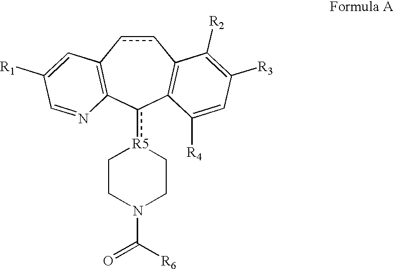

wherein R1 can be hydrogen (H) or halogen (e.g., F, Cl or Br)

R2, R3 and R4 are independently selected from H and halogen (e.g., F, Cl or Br) provided that at least one of R2, R3 and R4 is H;

“—” an optional bond;

R5 represents C when the optional bond to R5 is present and represents CH or N when the optional bond to R5 is absent;

R6 is selected from

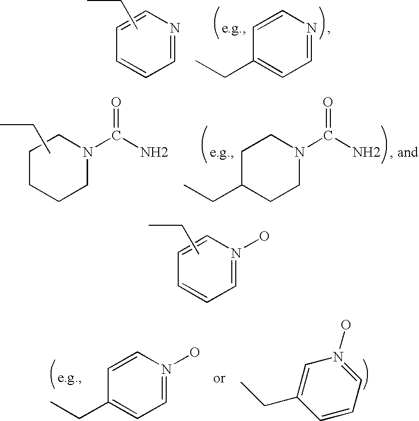

Preferably the optional bonds between C5 and C6 and on C11 (to R5), in Formula A, are absent. Preferably, R1 is halogen, more preferably Br. Representative compounds of Formula A include those wherein:

(1) R1 is halogen (preferably Br), R2 is halogen (preferably Br), R3 is halogen (preferably Cl), and R4 is H; or wherein

(2) R1 is halogen (preferably Br), R2 is H, R3 is halogen (preferably Cl) and R4 is halogen (preferably Br).

Preferably, the tricyclic amide is a compound of Formula A wherein R1 is halogen (preferably Br), R2 is H, R3 is halogen (preferably Cl) and R4 is halogen (preferably Br), the optional bond between C5 and C6 and to R5 is absent, R5 is CH and R6 is

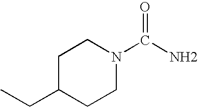

Most preferably the tricyclic amide is Formula 1:

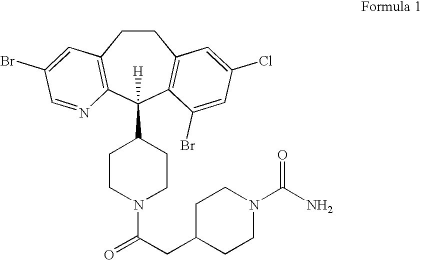

Those skilled in the art will appreciate that the tricyclic ring system is numbered according to the following illustration:

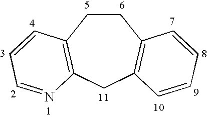

Other representative examples of the tricyclic amides of the present invention include but are by no means limited to:

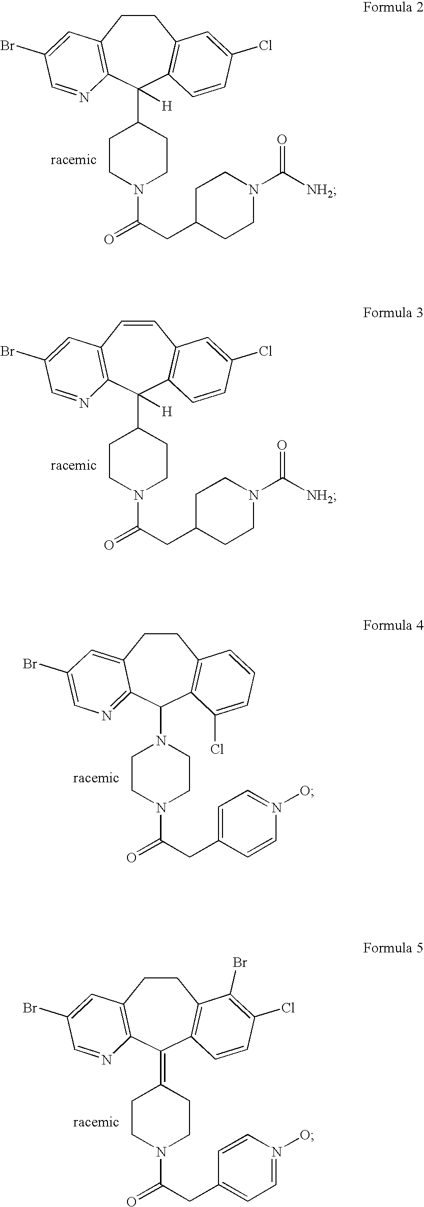

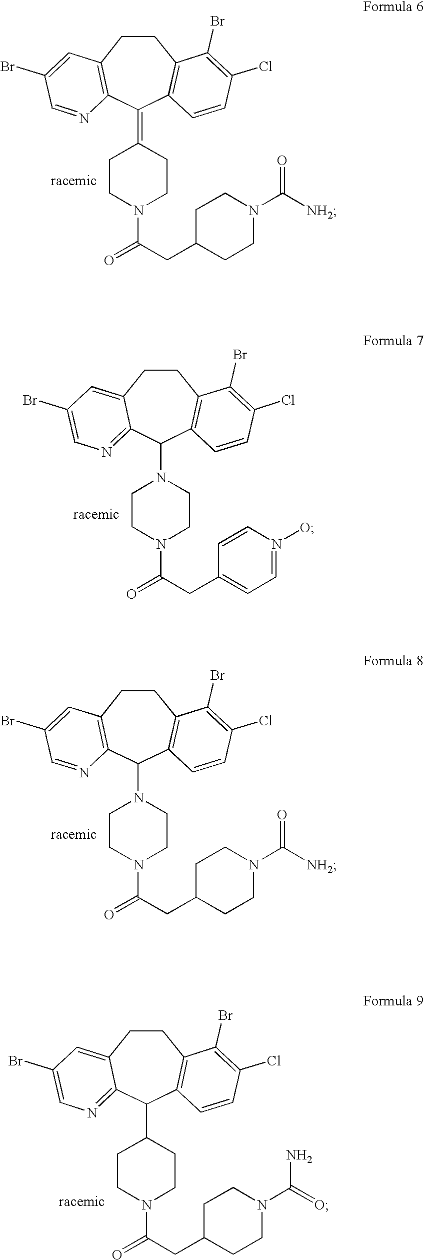

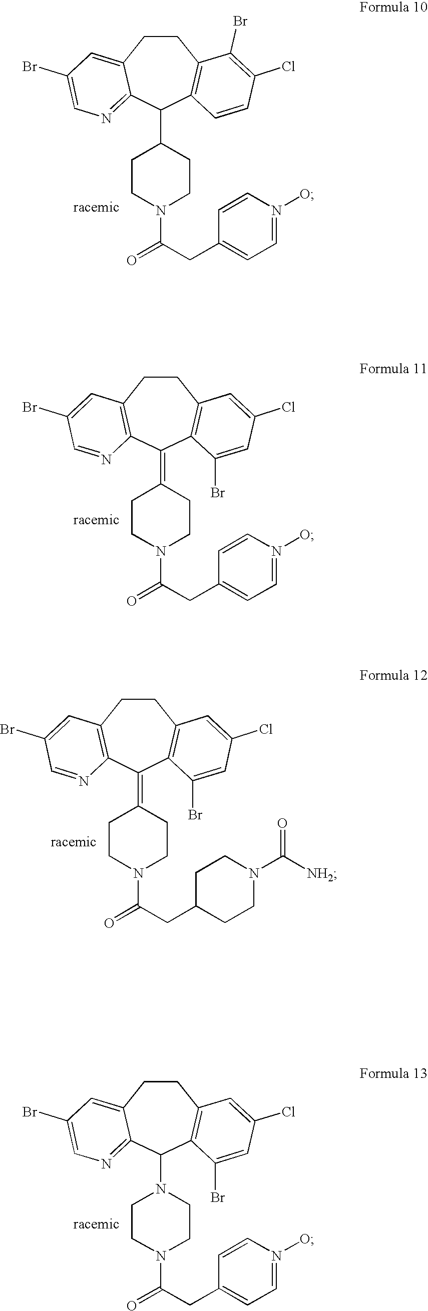

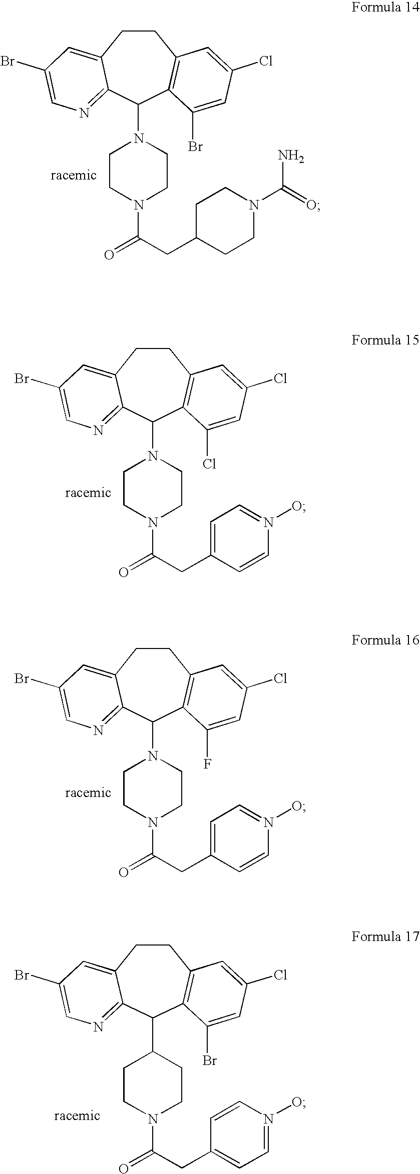

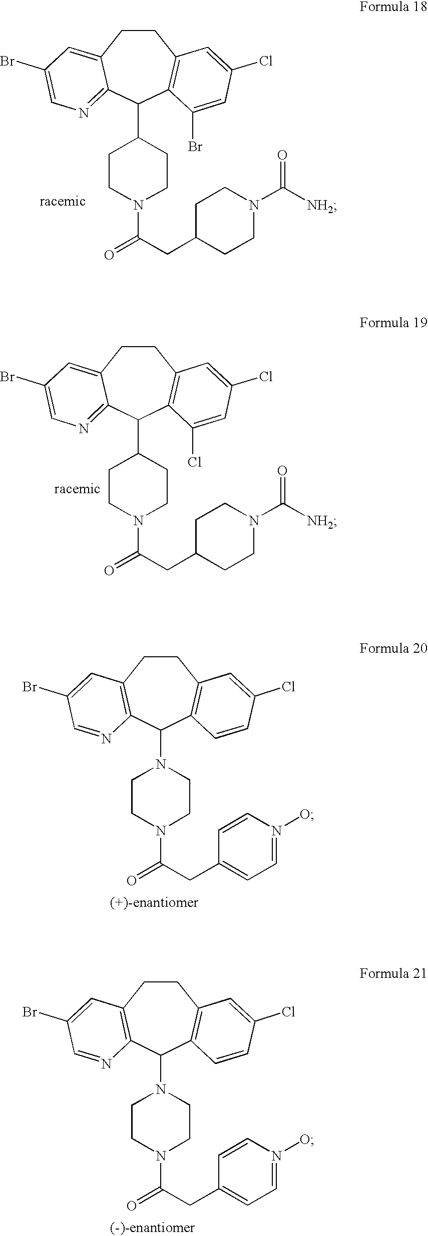

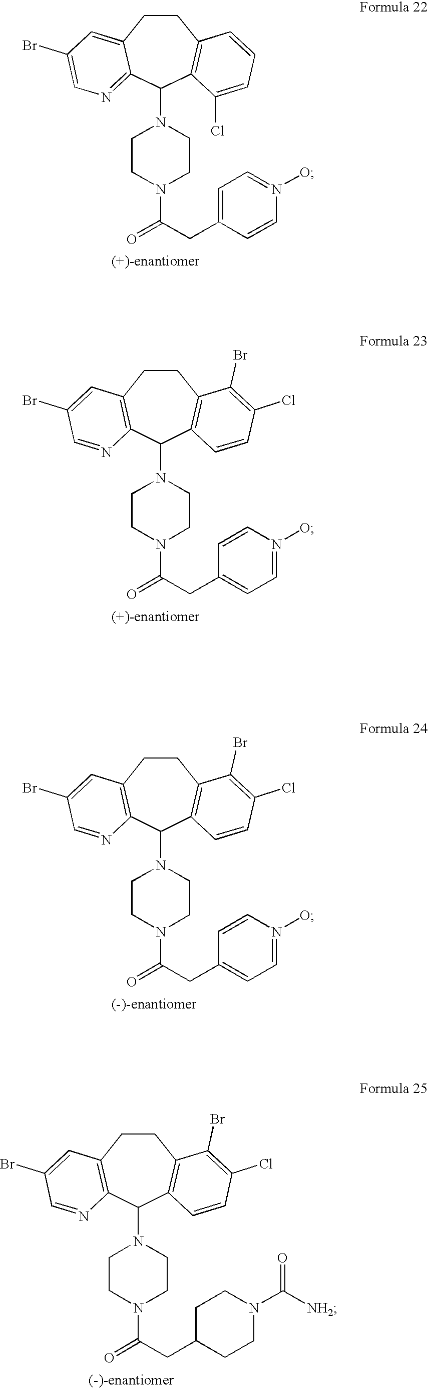

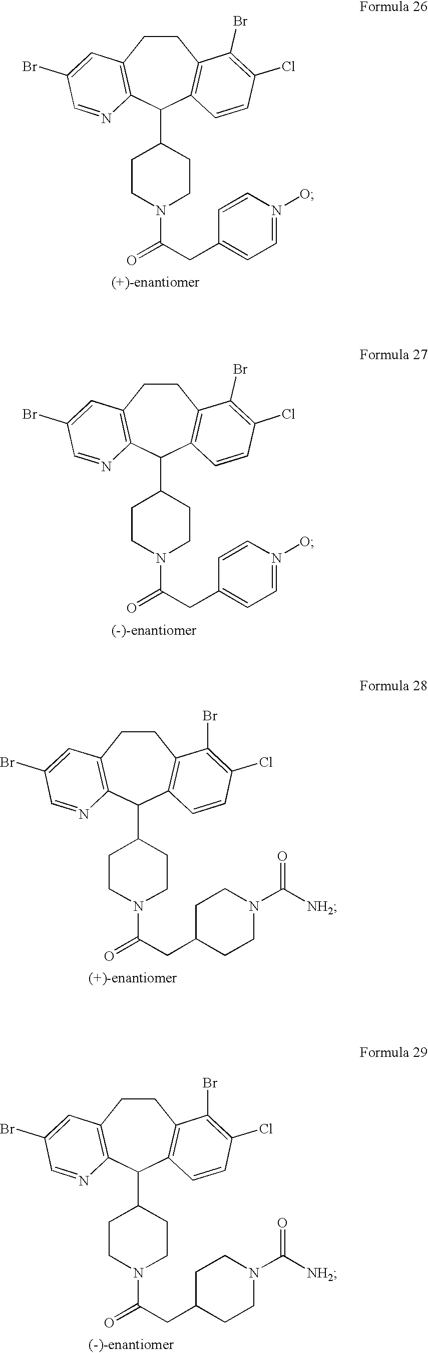

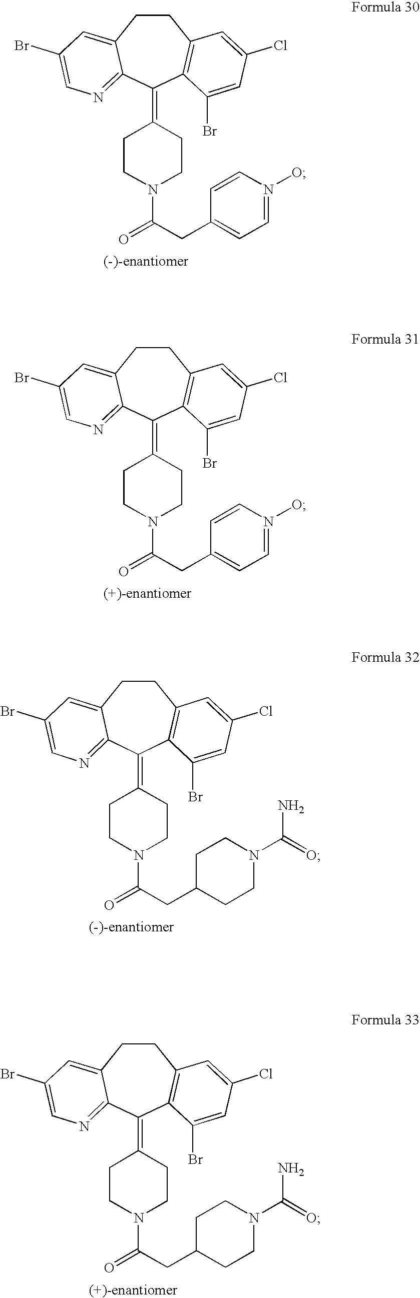

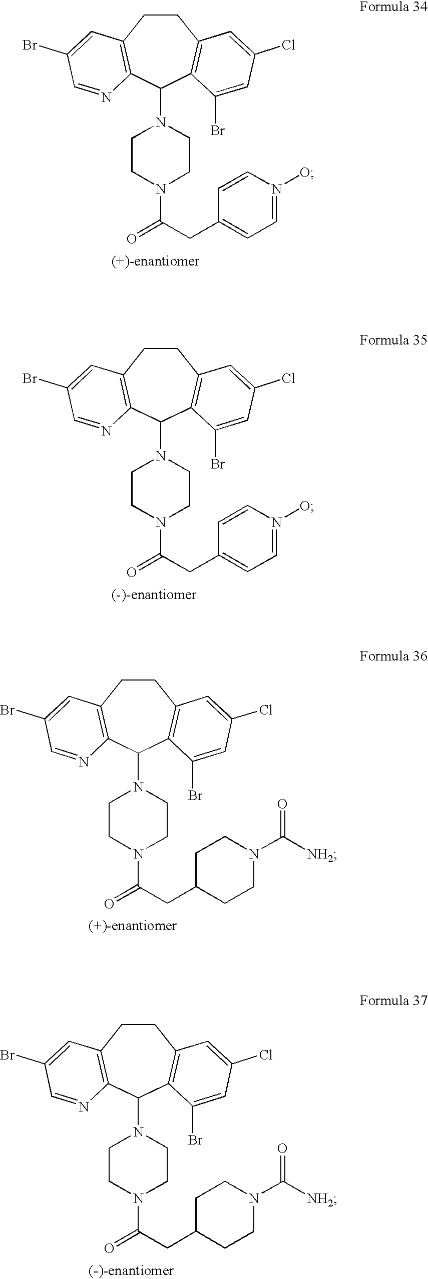

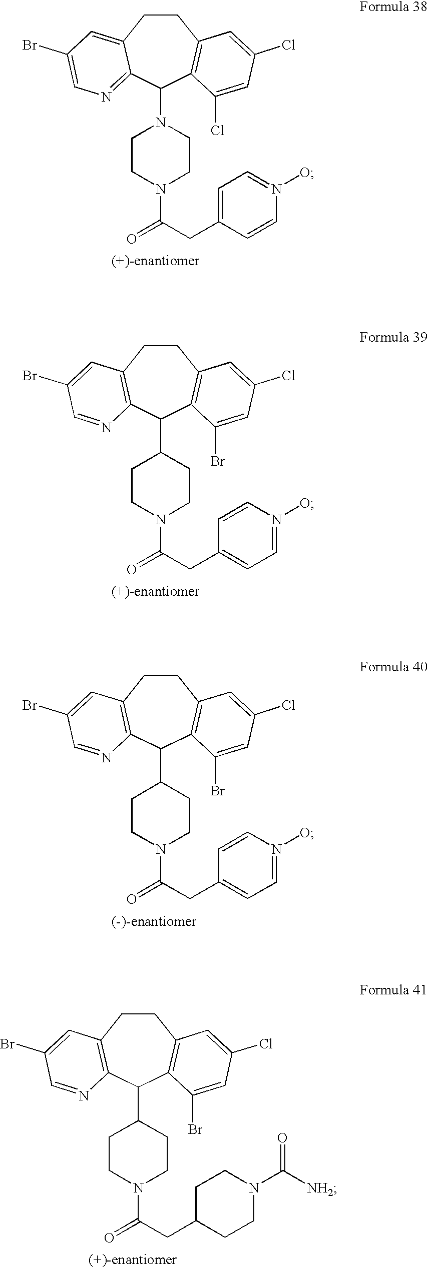

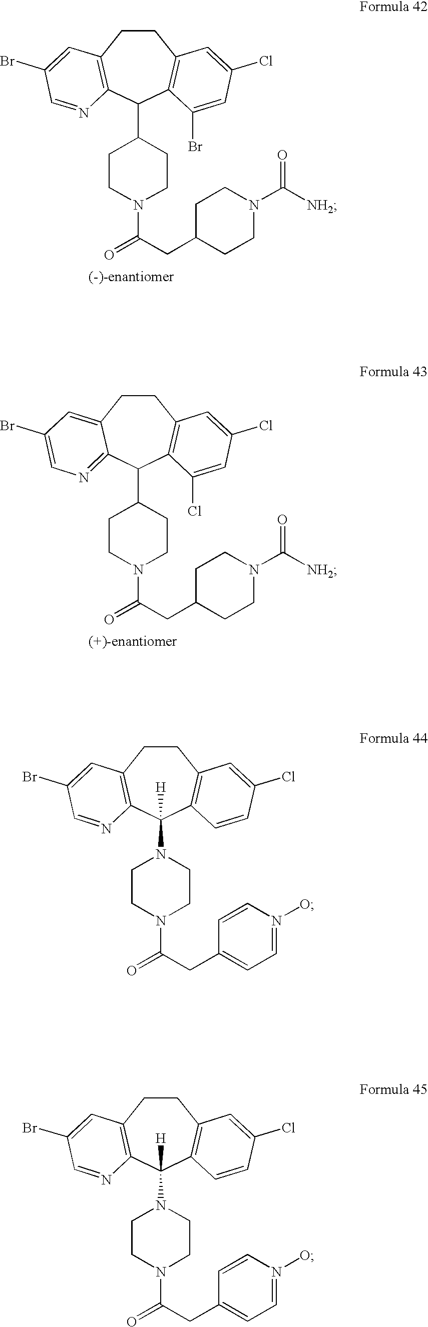

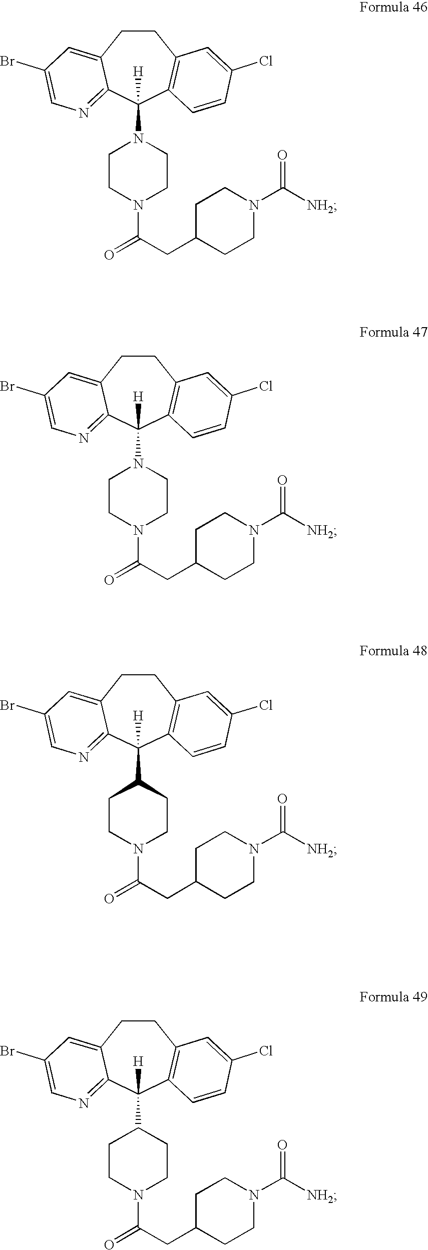

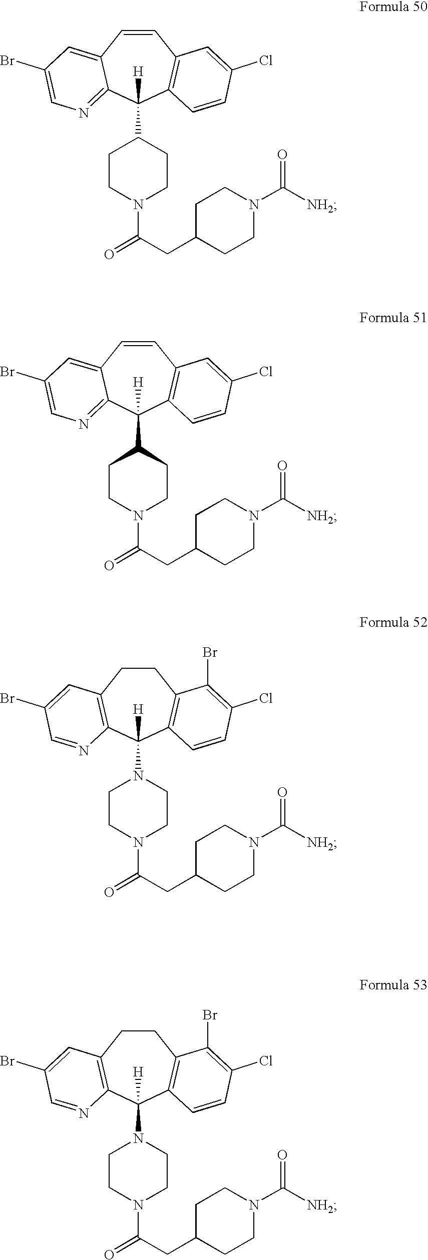

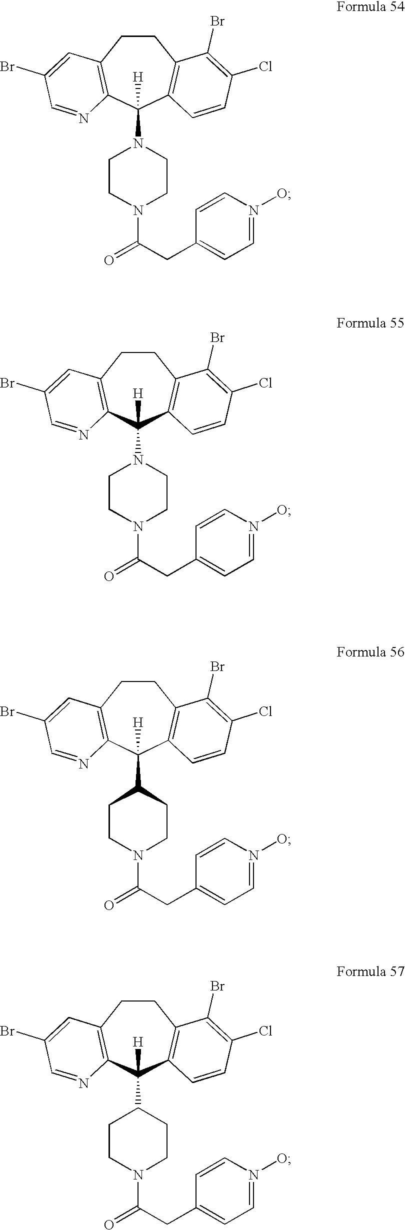

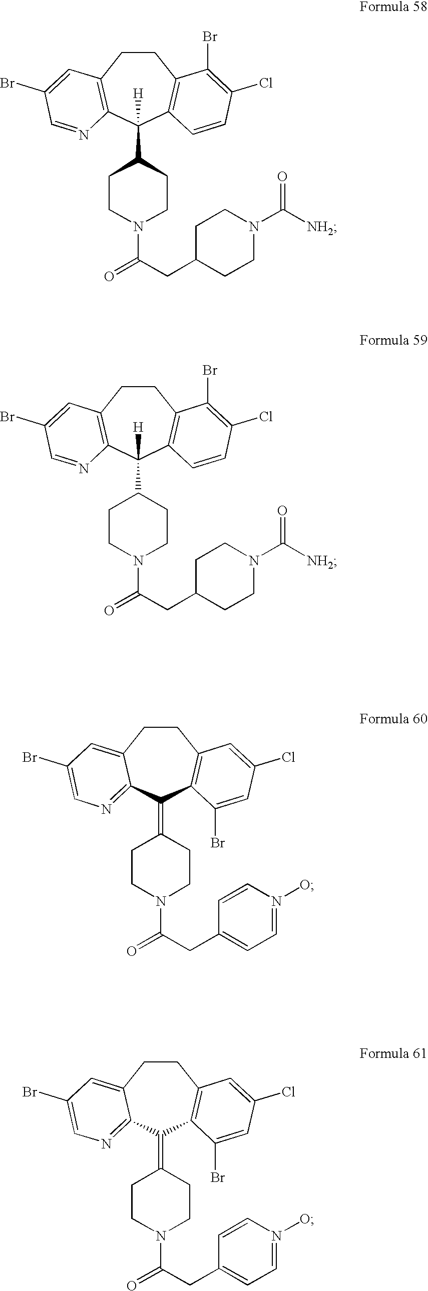

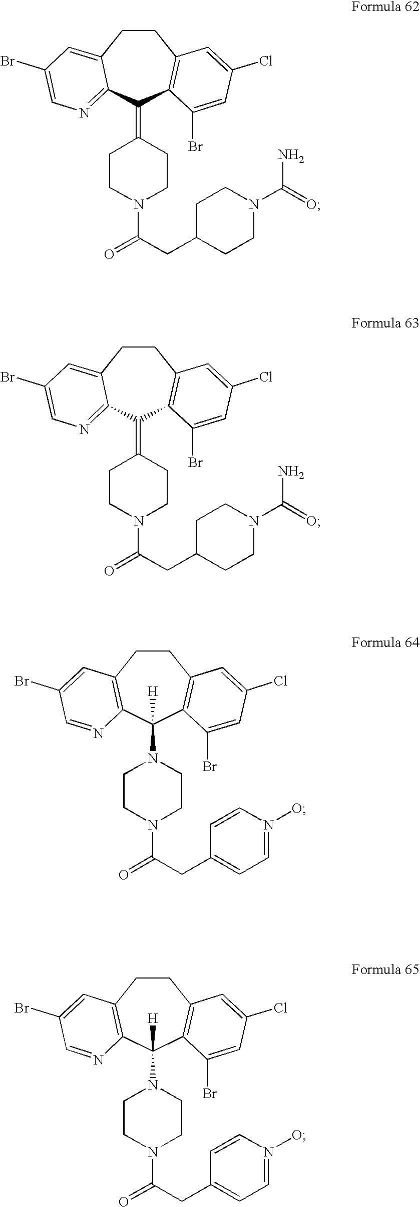

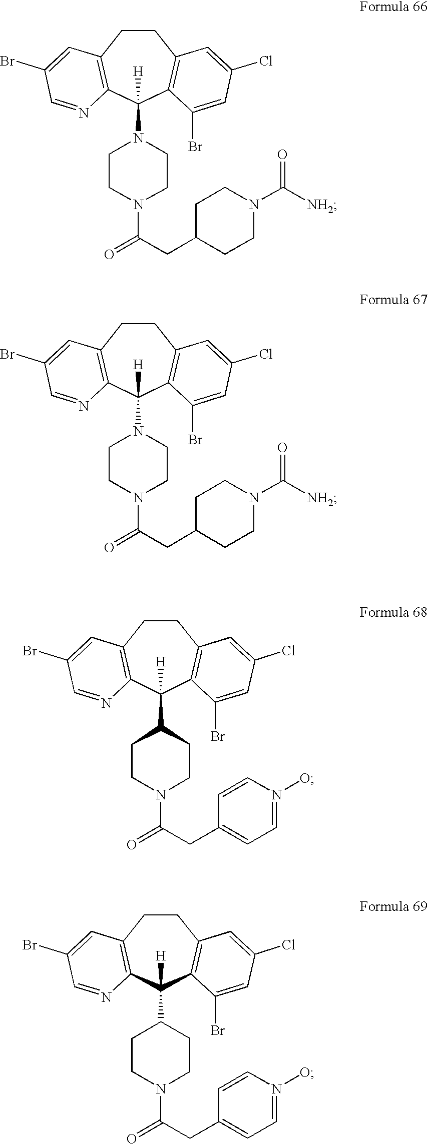

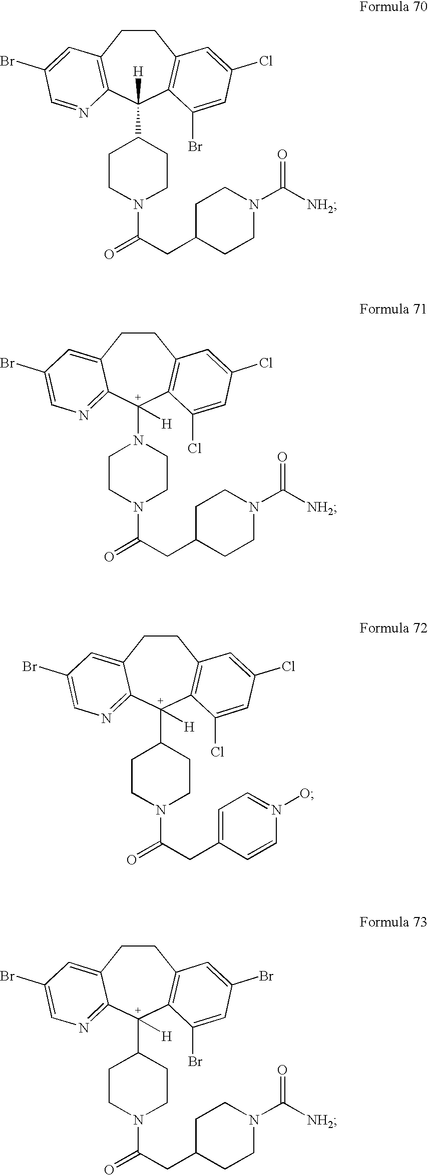

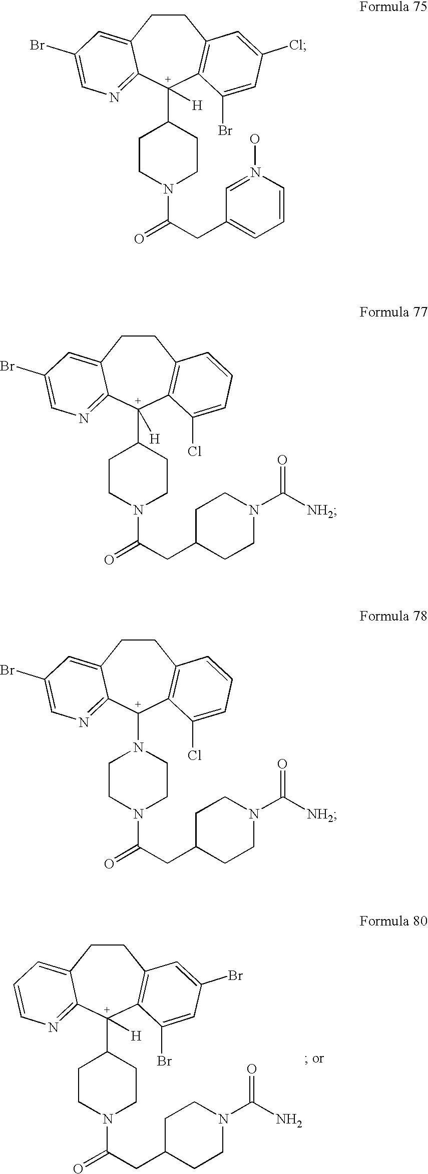

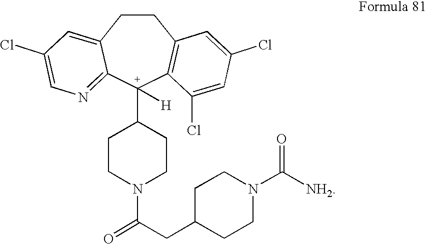

### Pharmaceutical Compositions, Dosage and Administration

The present invention includes pharmaceutical compositions comprising the tricyclic amide compounds of the invention (e.g., Formula 1) along with a pharmaceutically acceptable carrier.

The pharmaceutical compositions can be adapted for any mode of administration e.g., for oral, parenteral, e.g., subcutaneous (“SC”), intramuscular (“IM”), and intraperitoneal (“IP”) or topical administration or by inhalation (e.g., orally or intranasally). Preferably, tricyclic amide compounds of the invention (e.g., Formula 1) are administered orally (e.g., in a pill, capsule or tablet).

Such pharmaceutical compositions may be formulated by combining the tricyclic amide compounds (e.g., Formula 1) or an equivalent amount of a pharmaceutically acceptable salt thereof with a suitable, inert, pharmaceutically acceptable carrier or diluent that may be either solid or liquid.

Acceptable solid form preparations include powders, tablets, dispersible granules, capsules, cachets and suppositories. The powders and tablets may be comprised of, for example, from about 1 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co.; Easton, Pa.

Liquid form preparations include solutions, suspensions and emulsions. Such preparations include, for example, water or water-propylene glycol solutions for parenteral injection. Solid form preparations may be converted into liquid preparations shortly before use for either oral or administration. Parenteral forms to be injected intravenously, intramuscularly or subcutaneously are usually in the form of sterile solutions and may contain tonicity agents (e.g., salts or glucose), and buffers. Opacifiers may be included in oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, syrups, suspensions and emulsions.

Preferably, the pharmaceutical preparation is in a unit dosage form. In such form, the preparation is subdivided into suitably sized unit doses containing appropriate quantities of the active component, e.g., an effective amount to achieve the desired purpose.

In general, normal total serum calcium levels range from about 8.6 mg/dl to about 10.3 mg/dl (adult human), normal serum intact PTH levels range from about 10 pg/ml to about 65 pg/ml (adult human) and normal serum ionized Ca²⁺ levels range from about 4.64 to about 5.28 mg/dl (adult human) (Tietz Fundamentals of Clinical Chemistry (5^{th} Ed); W. B. Saunders Co.; New York; 2001). Methods for measuring serum Ca²⁺ or PTH are commonly known in the art. Although it is preferable for the methods of the present invention for treating calcium homeostatic disorders to lead to normal total serum Ca²⁺ and/or serum ionized Ca²⁺ and/or PTH levels in a subject, the present invention is not so limited. The present invention includes methods for treating calcium homeostatic disorders which lead to any detectable change in total serum Ca²⁺ levels or serum ionized Ca²⁺ levels or serum PTH levels toward a normal range or which lead to any degree of alleviation of symptoms associated with the particular disorder of calcium homeostasis.

### Combinations

The tricyclic amide compounds of the present invention may also be administered to a subject in association with a known, second substance which is useful for treating disorders of calcium homeostasis. Such substances include, but are not limited to, AMG073 (Goodman, et al., (2002) J. Am. Soc. Nephrol. 13:1017-1024 ), NPS467 (Nemeth, et al., (1998) Proc. Natl. Acad. Sci. 95:4040-4045), NPS568 (Nemeth, et al., (1998) Proc. Natl. Acad. Sci. 95:4040-4045), gadolinium, lanthanum, neomycin, Mg²⁺, 1,25-dihydroxyvitamin D (Delmez, et al., (1989) J. Clin. Invest. 83:1349-1355), calcitrol, paricalcitrol (Martin, et al., (1998) J. Am. Soc. Nephrol. 9:1427-1432), doxercalciferol (Frazao, et al., (2000) Am. J. Kidney Dis. 36:562-565), zoledronic acid (e.g., Zometa; Davidson, (2001) Am. J. Health Syst. Pharm. 58 Suppl. 3:S8-15), calcitonin, alfacalcidol and oxacalcitriol. Each of these substances are well known in the art.

The tricyclic amide compounds of the invention may be formulated with the second substance into a single composition or into two or more separate compositions for simultaneous consumption. Alternatively, a tricyclic amide compound of the invention may be administered to a subject at a different time than when the second substance is administered; for example, each administration may be given non-simultaneously at several intervals over a given period of time.

### EXAMPLES

### Example 1

### Effect of the Compound of Formula 1 on Calcium Homeostasis and PTH

A study was undertaken to assess the potential nephrotoxicity of the compound of Formula 1 in rats. The rats were 6 week old females weighing 120.7 to 173.5 g at dosing initiation.

The study design is summarized below in Tables 1-3:

TABLE 1
One-Month Exploratory Nephrotoxicity Study of the Compound of
Formula 1 in Female Rats: Study Design.

| | | | | Dose | | No. Rats | |
|---|---|---|---|---|---|---|---|
| | | Total Daily | Dose | Suspension | No. | Bled for | Duration of |
| | Test/control | Dose | Volume | Conc. | Female | Plasma | Dosing |
| Group | Article | (mg/kg) | (mg/kg) | (mg/kg) | Rats | Analysis¹ | (days) |
| Control | Methyl- | 0 | 5 | 0 | 20 | 20² | 3, 7, 14 or |
| | cellulose | | | | | | 30 |
| | (0.4%; | | | | | | |
| | aqueous) | | | | | | |
| Test | Formula 1 | 180 | 5 | 36 | 20 | 20² | 3, 7, 14 or |
| | | | | | | | 30 |

¹2 hours post-dose.
²Blood samples were collected from five rats/group/time point on Days 2, 6, 13 and 29.

TABLE 2
One-Month Exploratory Nephrotoxicity Study of the Compound of
Formula 1 in Female Rats: Observations and Measurements.

| Investigation | Performed | Investigation | Performed |
|---|---|---|---|
| Viability and | Daily beginning | Urinalysis/Urine | Days 2, 6, 13 and 29 |
| Clinical | Week-1 | Chemistry | |
| Observations | | | |
| Body Weight | Weekly beginning | Organ Weights¹ | Days 2, 6, 13 and 29 |
| | Week-1, and days of | | |
| | randomization and | | |
| | terminal sacrifice | | |
| Food Consumption | Weekly beginning | Necropsy | Days 2, 6, 13 and 29 |
| | Week-1 | (Macroscopic | |
| | | Observations) | |
| Water Consumption | Days 2, 6, 13 and 29 | Histopathology | Days 2, 6, 13 and 29 |
| | | (Microscopic | |
| | | Observations)² | |
| Plasma analysis for | Days 2, 6, 13 and 29 | Ultrastructural | Days 13 and 29 |
| Formula 1 | (2 hours post-dose) | Pathology³ | |
| Serum Chemistry | Days 2, 6, 13 and 29 | | |

¹Kidneys only.
²Kidneys and thyroid/parathyroid glands only.
³Parathyroid glands only.

TABLE 3
One-Month Exploratory Nephrotoxicity Study of the Compound of
Formula 1 in Female Rats: Assignment of Rats for Plasma Analysis, Serum
Chemistry, Urinalysis, Urine Chemistry and Histopathology Evaluations.

| Animal | | Blood (ml- | | | Urinalysis/ | |
|---|---|---|---|---|---|---|
| Order/Group/ | Water | site of | Plasma | Serum | Urine | Histo- |
| Sacrifice Day | Consumption | bleeding) | Analysis (ml)¹ | Chemistry | Chemistry | pathology |
| 1^{st} | ✓ | 4/aorta | 1.5 | ADH | Std² | — |
| 2^{nd} | ✓ | 4/aorta | 1.5 | ADH | Std² | — |
| 3^{rd} | ✓ | 4/aorta | 1.5 | ADH | Std² | — |
| 4^{th} | ✓ | 4/aorta | 1.5 | ADH | Std² | — |
| 5^{th} | ✓ | 4/aorta | 1.5 | ADH | Std² | — |
| 6^{th} | ✓ | 4/aorta | — | std, PTH, | Std² | K, PT |
| | | | | vitDs, free | | |
| | | | | Ca | | |
| 7^{th} | ✓ | 4/aorta | — | std, PTH, | Std³ | K, PT |
| | | | | vitDs, free | | |
| | | | | Ca | | |
| 8^{th} | ✓ | 4/aorta | — | std, PTH, | Std³ | K, PT |
| | | | | vitDs, free | | |
| | | | | Ca | | |
| 9^{th} | ✓ | 4/aorta | — | std, PTH, | Std³ | K, PT |
| | | | | vitDs, free | | |
| | | | | Ca | | |
| 10^{th} | ✓ | 4/aorta | — | std, PTH, | Std³ | K, PT |
| | | | | vitDs, free | | |
| | | | | Ca | | |
| ¹Blood samples were collected from five rats/group/time point on Days 2, 6, 13 and 29. | | | | | | |
| ²Standard panel of urine chemistry except creatinine clearance (collected at room temperature). | | | | | | |
| ³Standard panel of urine chemistry plus cAMP, GGT and NAG (collected and chilled at approximately 4° C.). | | | | | | |
| ADH: Antidiuretic hormone | | | | | | |
| cAMP: Cyclic adenosine monophosphate | | | | | | |
| free Ca: Free calcium | | | | | | |
| GGT: Gamma-glutamyl transpeptidase | | | | | | |
| K: Kidneys | | | | | | |
| NAG: N-acetyl glucosaminidase | | | | | | |
| PT: Parathyroid glands | | | | | | |
| PTH: Parathyroid hormone | | | | | | |
| vitDs: 1, 25-dihydroxy vitamin D and 25-hydroxy vitamin D | | | | | | |
| Std.: Standard Panel of Serum Chemistry: Blood was collected at necropsy from the abdominal aorta. For the ten rats sacrificed at each timepoint, serum from five rats were analyzed for the standard chemistry profile and antidiuretic hormone (ADH). Serum from the remaining five rats were analyzed for parathyroid hormone, 1, 25-dihydroxy vitamin D, 25-hydroxy vitamin D and free calcium. Blood (2-4 ml/rat) was collected into a 4 ml draw red/gray top (no anticoagulant) serum separator | | | | | | |
| #tube and the following parameters were measured: hemolysis; lipemia; icterus; glucose; urea nitrogen; creatinine; alkaline phosphatase; total protein; albumin; globulin (calculated); albumin/globulin ratio (calculated); sodium; potassium; chloride; calcium (total); free calcium; phosphorus; parathyroid hormone; vitamin D; antidiuretic hormone (ADH); 1, 25-dihydroxy vitamin D; 25-hydroxy vitamin D. | | | | | | |
| Std.: Standard Panel of Urine Chemistry: Urine was collected and chilled at approximately 4° C. from all rats by cage run-off during 24 hours preceding each scheduled necropsy. For freshly voided samples, at least 2 ml of urine were collected from each rat. For 24-hour samples, urine was collected from each rat for the remaining 24 hours after voided sample collection. All urine samples were collected in 50 ml centrifuge tubes, chilled and stored at approximately 4° C. before | | | | | | |
| #analysis. Unused voided samples were combined with the respective 24-hour samples for urinalysis. The following parameters were measured in freshly voided samples: color; clarity; pH; protein; glucose; ketones; bilirubin; blood; urobilinogen; microscopic analysis. For 24-hour samples: sodium; potassium; chloride; calcium (total); phosphorus; creatinine clearance; volume; osmolality. | | | | | | |

TABLE 4
Mean calcium and phosphorus excretion (g/kg/24 hours).

| | Day | Day | Day | Day | Day | Day | Day | Day |
|---|---|---|---|---|---|---|---|---|
| | 2 | 2 | 6 | 6 | 13 | 13 | 29 | 29 |
| Formula 1 | Ca | P | Ca | P | Ca | P | Ca | P |
| 0 mg/kg | 7.30 | 57.93 | 5.55 | 51.60 | 9.56 | 37.8 | 10.88 | 39.43 |
| 180 mg/kg | 5.65 | 55.86 | 32.99 | 13.54 | 20.69 | 12.11 | 19.24 | 7.30 |

TABLE 5
Mean serum PTH (ng/ml).

| Formula 1 | Day 2 | Day 6 | Day 13 | Day 29 |
|---|---|---|---|---|
| 0 mg/kg | 94 | 54 | 89 | 90 |
| 180 mg/kg | 60 | 51 | 68 | 44 |

TABLE 6
Mean serum ADH (pg/ml).

| Formula 1 | Day 2 | Day 6 | Day 13 | Day 29 |
|---|---|---|---|---|
| 0 mg/kg | 193 | 97 | 115 | 619 |
| 180 mg/kg | 350 | 513 | 547 | 616¹ |

¹At this timepoint, water consumption had increased, which would result in less stimulus for ADH production.

TABLE 7
Mean urine volume (ml/kg/24 hours) and osmolality (mOsm/kg).

| | Day | Day | Day | Day | Day | Day | Day | Day |
|---|---|---|---|---|---|---|---|---|
| | 2 | 2 | 6 | 6 | 13 | 13 | 29 | 29 |
| Formula 1 | Vol. | Osmo | Vol. | Osmo | Vol. | Osmo | Vol. | Osmo |
| 0 mg/kg | 75 | 1541 | 71 | 1573 | 100 | 1176 | 84 | 1073 |
| 180 mg/kg | 124 | 646 | 195 | 646 | 177 | 655 | 166 | 606 |

Within 6 days of dosing at 180 mg/kg, calcium excretion had increased in the urine and phosphorus excretion was decreased. Furthermore, a decrease in serum PTH was observed in rats dosed with the compound of Formula 1 and, within 28 days, 5 out of 5 rats evaluated had lower PTH serum concentrations than did the control rats. Decreases in urine osmolality, increases in urine volume and increases in antidiuretic hormone (ADH) concentrations suggest the development of nephrogenic diabetes insipidus (failure of kidneys to concentrate urine in response to ADH).

These data also strongly suggest that structurally related tricyclic amide compounds (e.g., formulas 2-81or those disclosed in U.S. Pat. No. 5,719,148 or in U.S. Pat. No. 5,874,442) would exhibit in vivo properties which are similar to those described above for the compound of Formula 1. Specifically, the structurally related tricyclic amide compounds should also be useful for treating calcium homeostatic disorders such as hypercalcemia.

Without being bound by a single theory, agonism of the CaSR in the parathyroid gland of a subject (e.g., by administration of Formula 1) may lead to lower serum parathyroid hormone levels which in turn may lead to lower Ca²⁺ serum levels. The parathyroid cells may decrease PTH secretion by fusing secretory granules containing PTH with lysosomes; this would lead to PTH degradation. Again, without being bound by a single theory, at the kidney level, the Calcium Ion Sensing Receptor may help regulate calcium ion reabsorption. Calcium regulation at the kidney may be independent of parathyroid hormone (Brown, et al., (1995) N. Engl. J. Med. 333(4):234-240). Under this theory, when the Calcium Ion Sensing Receptor is activated by increased calcium ions in the plasma, there is a diminished uptake of calcium from the filtrate, resulting in increased calciuria.

### Example 2

### Micrographic Analysis of Rat Parathyroid Glands

Microscopic evaluation of the parathyroid glands of rats dosed with 180 mg/kg Formula 1 revealed cytoplasmic vacuolation.

Electron photomicrographs of the right parathyroid gland from two control rats sacrificed on Day 13 (Nos. 26F and 29F), two control rats sacrificed on Day 29 (Nos. 39F and 40F) and two Formula 1-dosed rats sacrificed on Day 29 (Nos. 76F and 77F) were examined for ultrastructural changes. The majority of parathyroid chief cells from the two Formula 1-dosed rats sacrificed on Day 29 had less conspicuous cell borders with fewer, less distinctive cytoplasmic projections. This suggests that the gland from which the cells were obtained was less active in PTH production/secretion. Consistent with the light microscopic appearance, the cytoplasm contained numerous, generally membrane-bound, vesicles, approximately 0.2-3 μm in diameter, that were generally electron lucent with occasional irregular, variably electron-dense material. The number of myeloid bodies and fat globules was minimally increased. A single rounded parathyroid epithelial cell in animal No. 77F appeared apoptotic with condensed chromatin and intact mitochondria and contained numerous vesicles similar to those previously described. The apoptotic cell is consistent with the single cell necrosis observed at the light microscopic level. This too suggests that the gland from which the cells were obtained was less active in PTH production/secretion.

Considering the increased number of vesicles and myeloid bodies in the Formula 1-dosed rats, some vesicles may have fused with lysosomes (crinophagy) which would lead to a decrease in PTH secretion. There was no evidence of exhaustion vacuoles. Additionally, there were no changes indicating chronic stimulation such as increased rough endoplasmic reticulum and prominent Golgi apparatus.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, Genbank submission accession numbers, product descriptions, and protocols are cited throughout this application, the disclosures of which are incorporated herein by reference in their entireties for all purposes.

We claim:

1. A method for treating or preventing a disorder of calcium homeostasis in a subject comprising administering, to the subject, a compound according to the following formula: 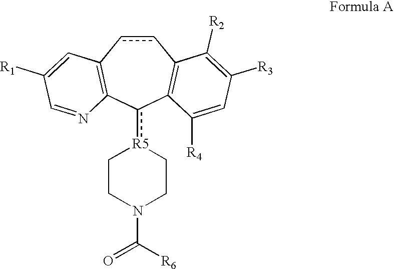
wherein R1 can be hydrogen or halogen
R2, R3 and R4 are independently selected from H and halogen provided that at least one of R2, R3 and R4 is H;
— represents an optional bond;
R5 represents C when the optional bond to R5 is present and represents CH or N when the optional bond to R5 is absent; and
R6 is selected from

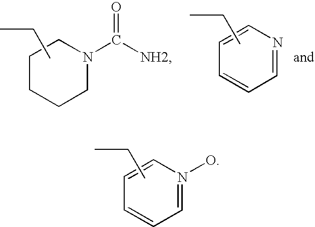

2. The method of claim 1 wherein the compound of Formula A comprises a formula selected from the group consisting of:

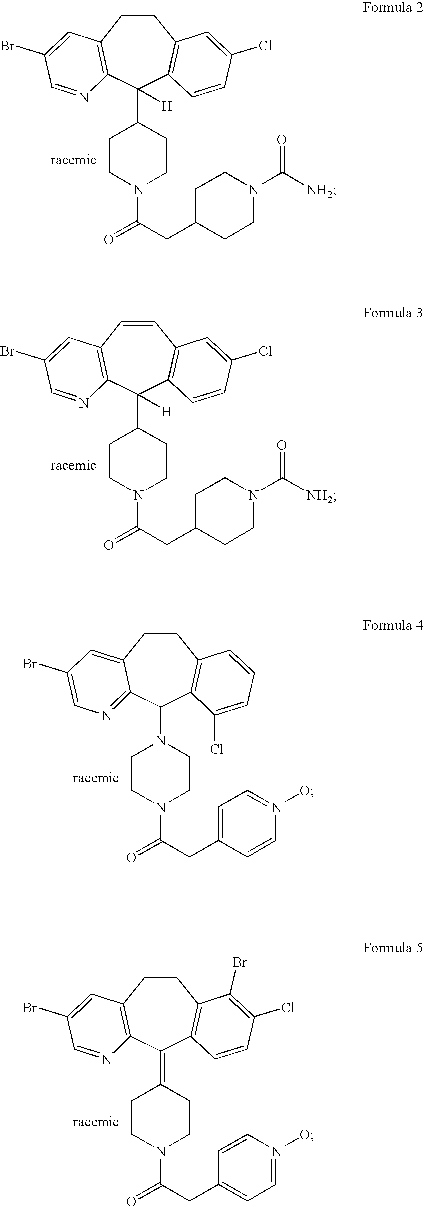

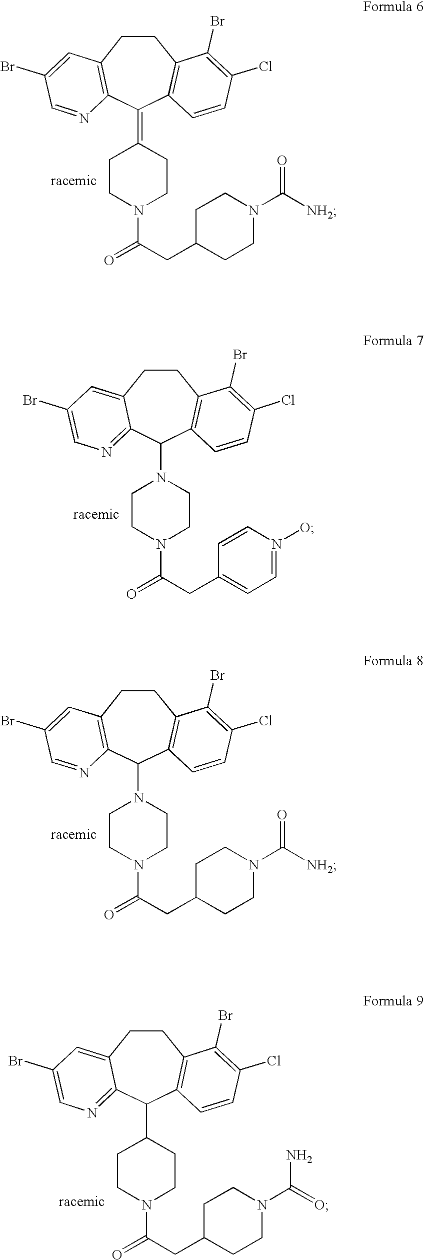

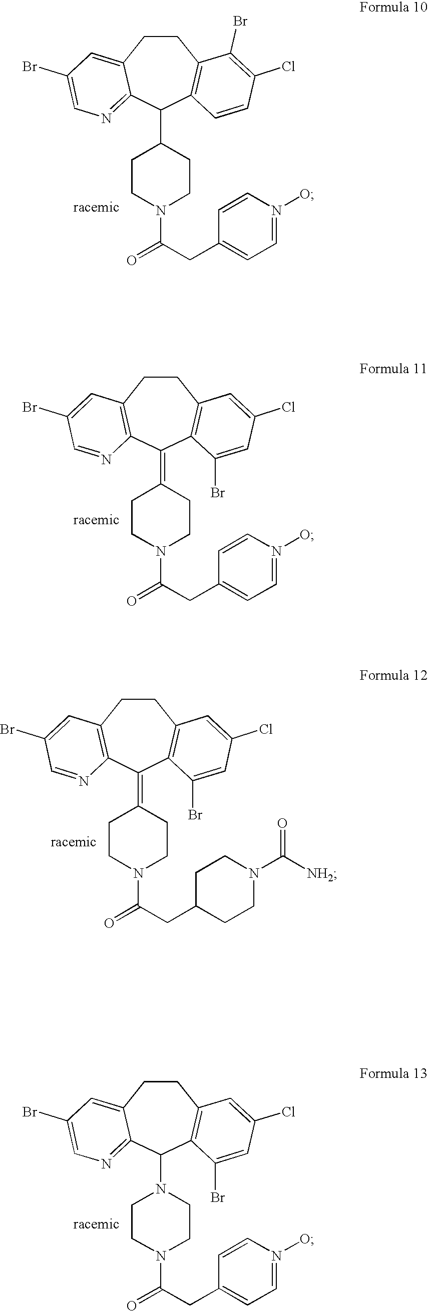

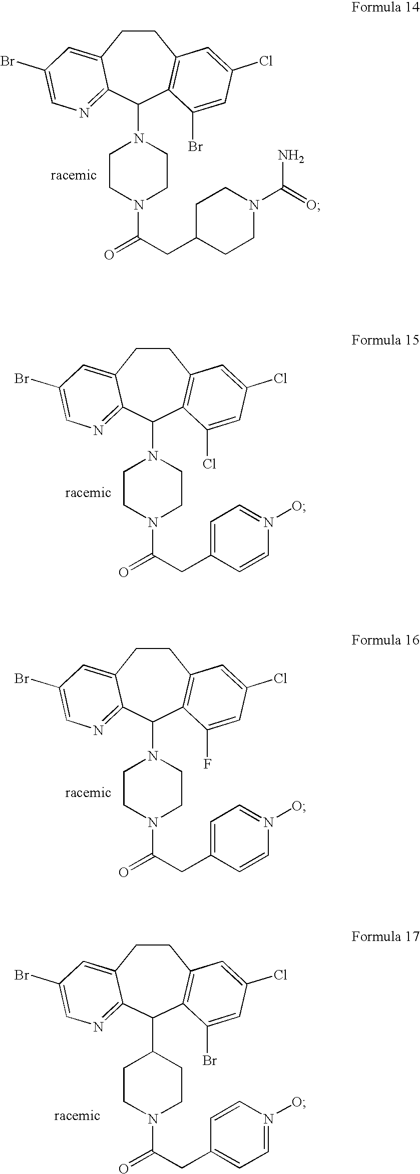

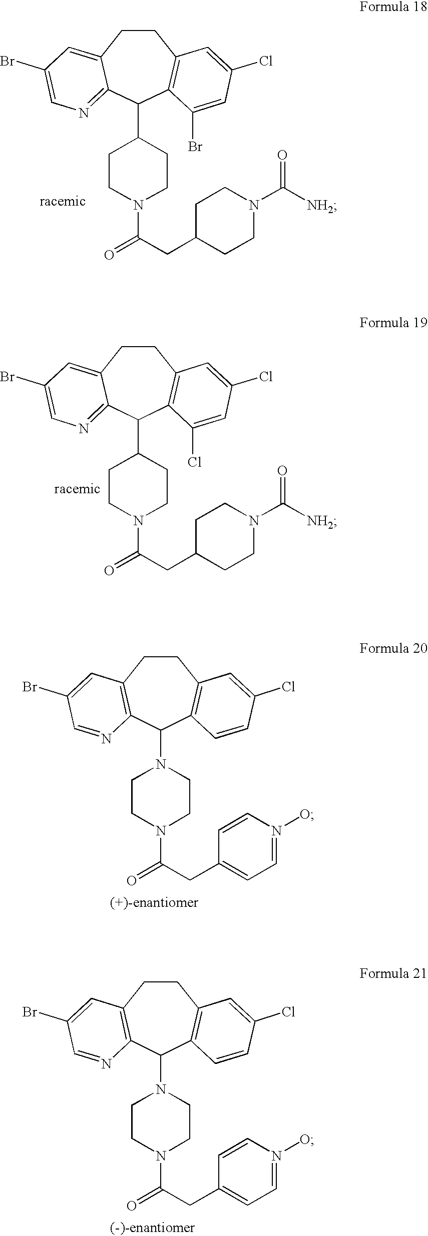

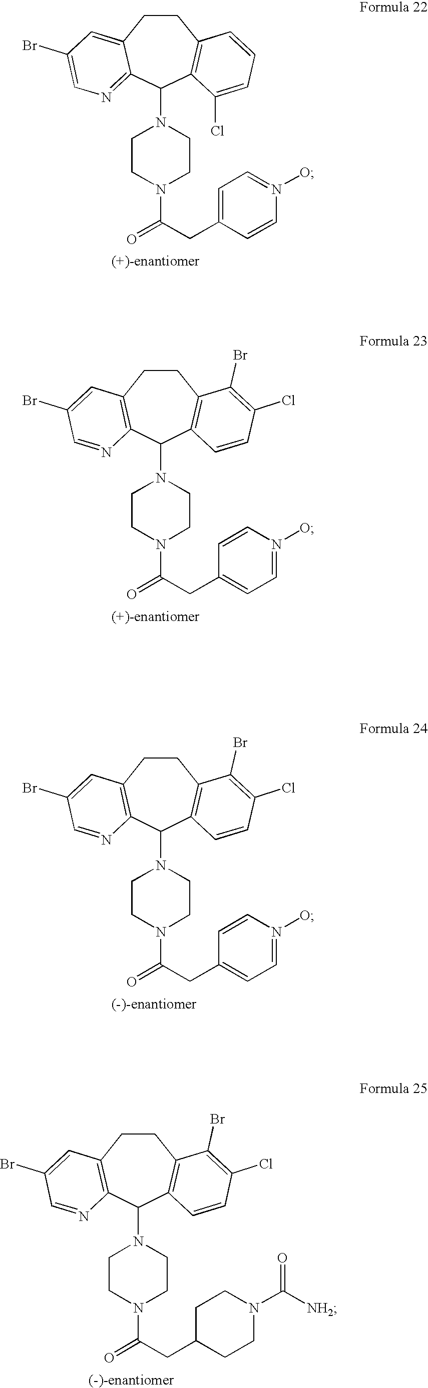

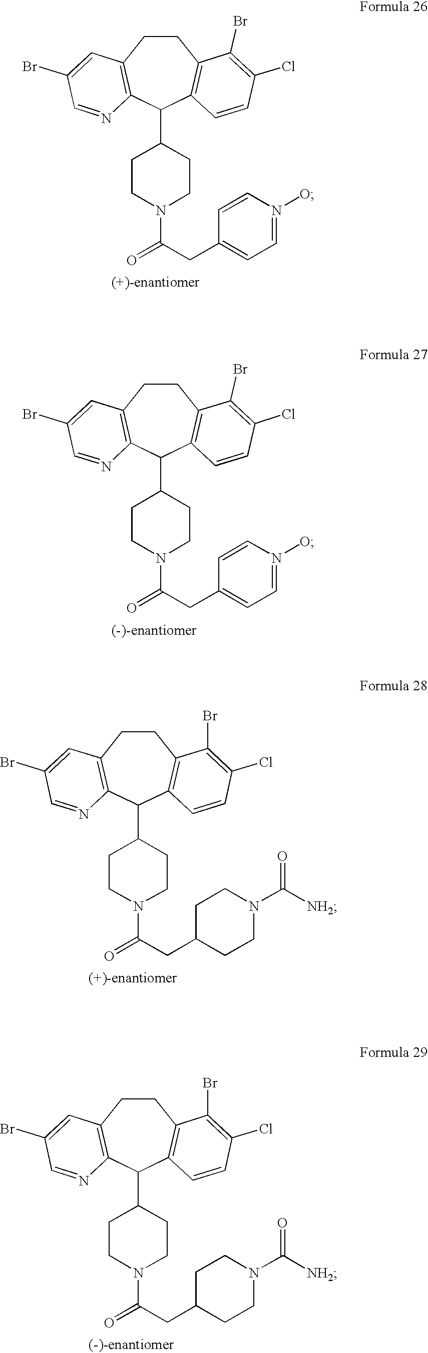

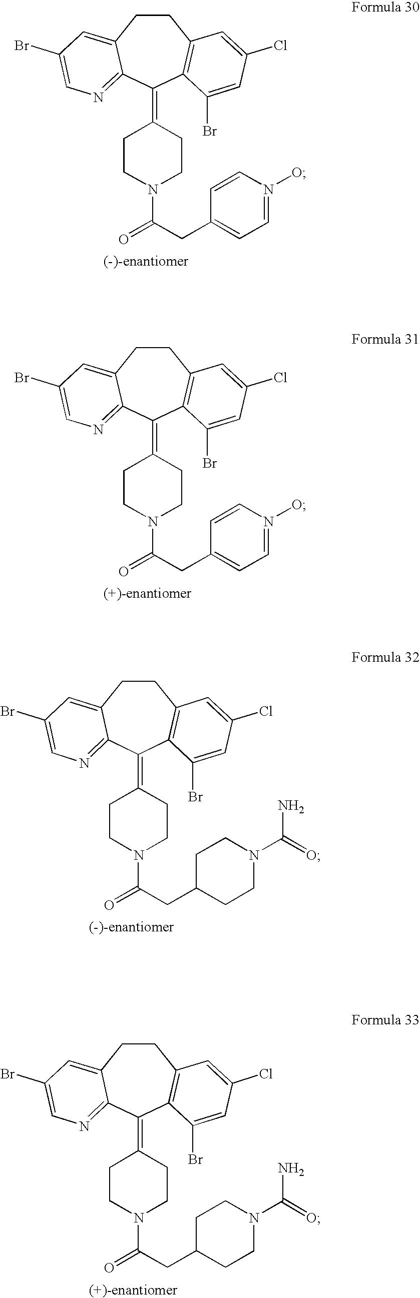

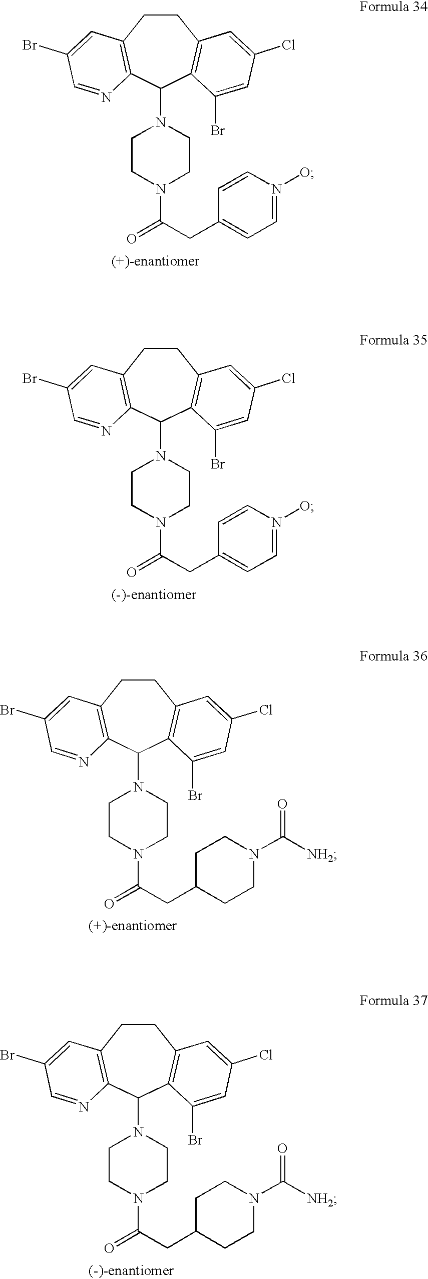

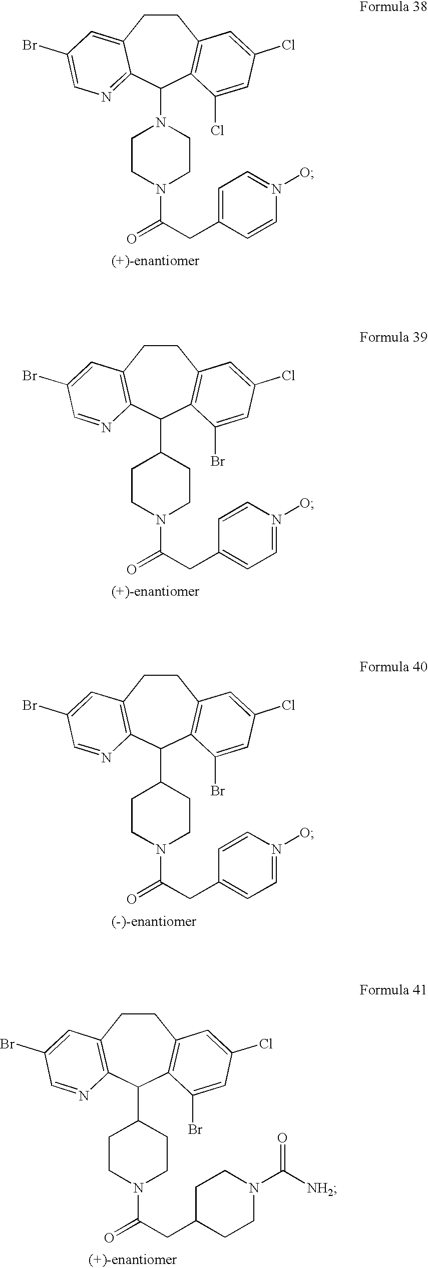

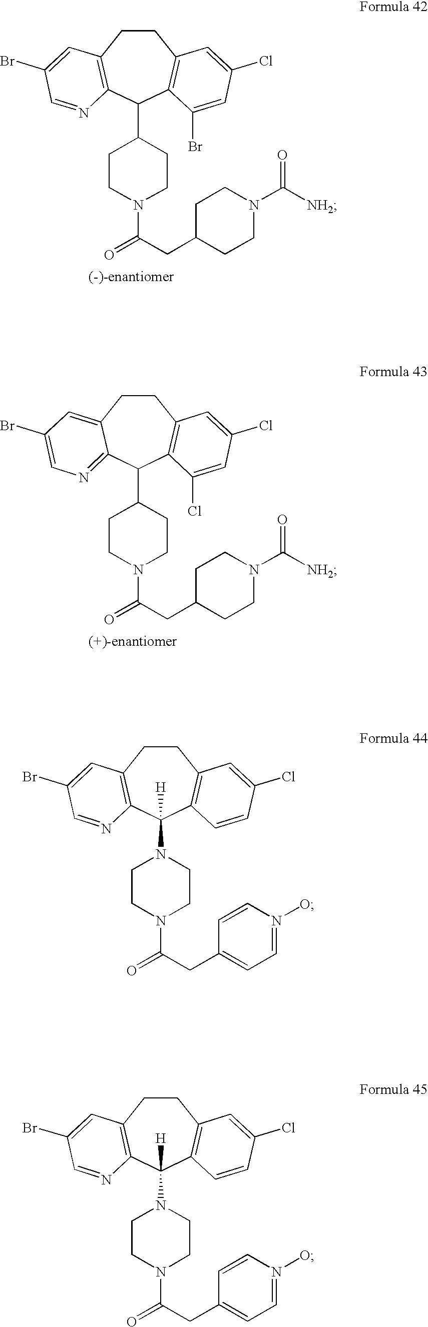

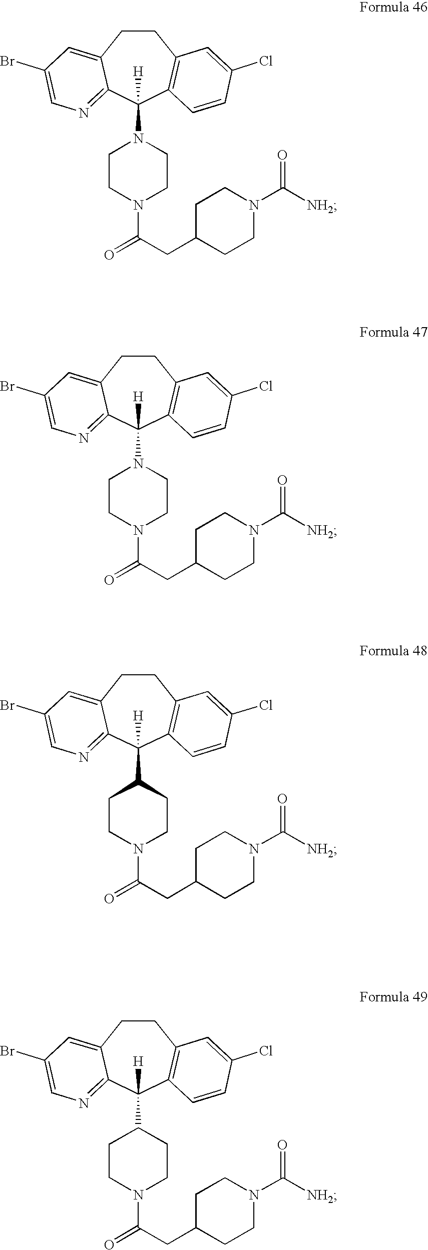

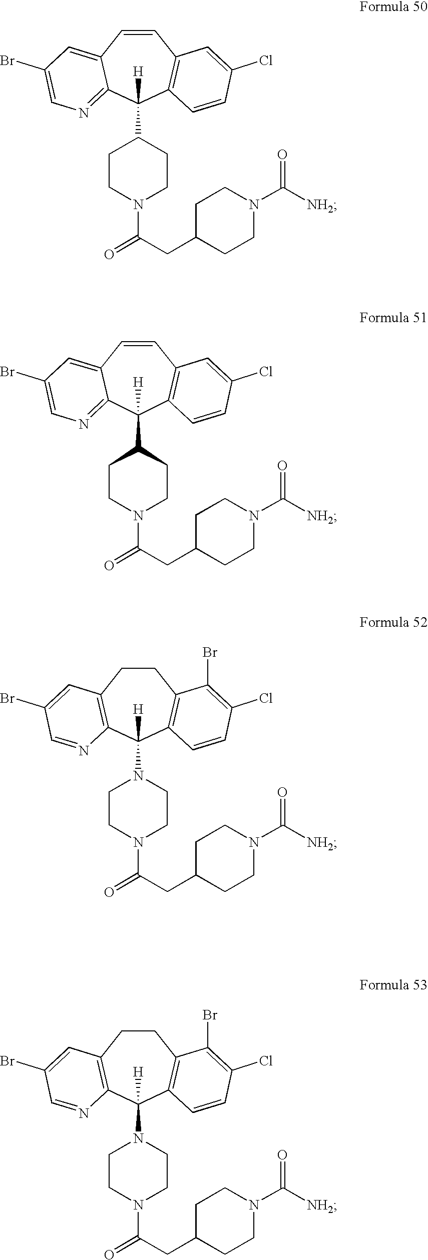

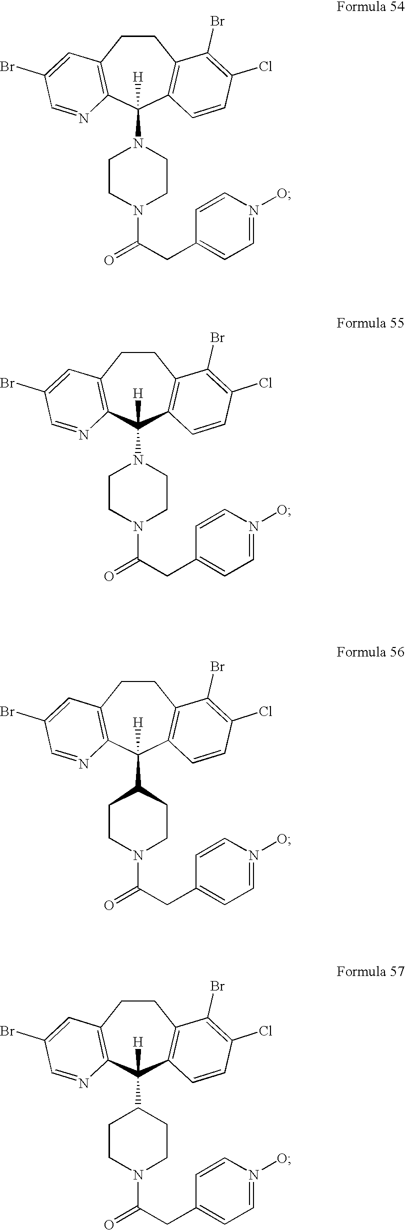

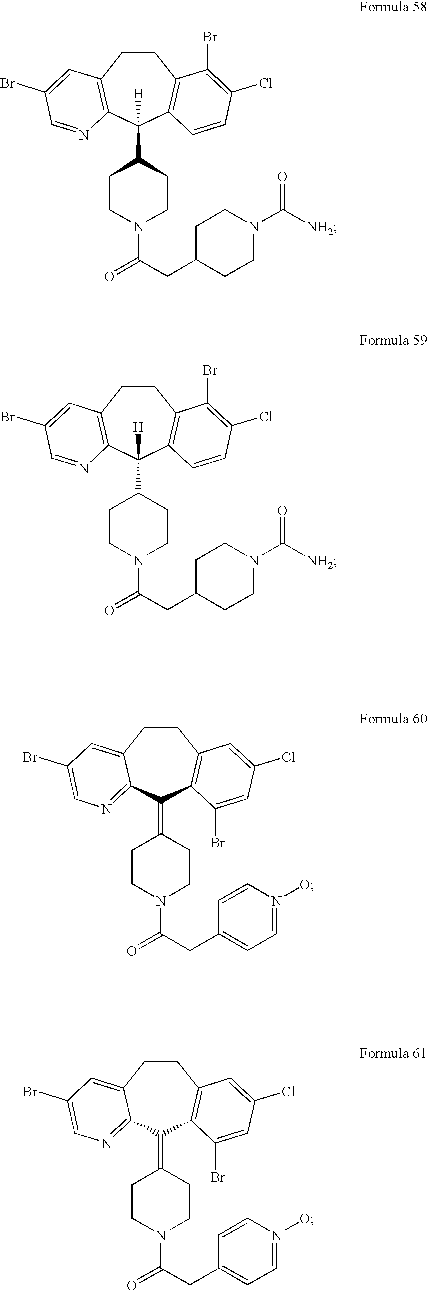

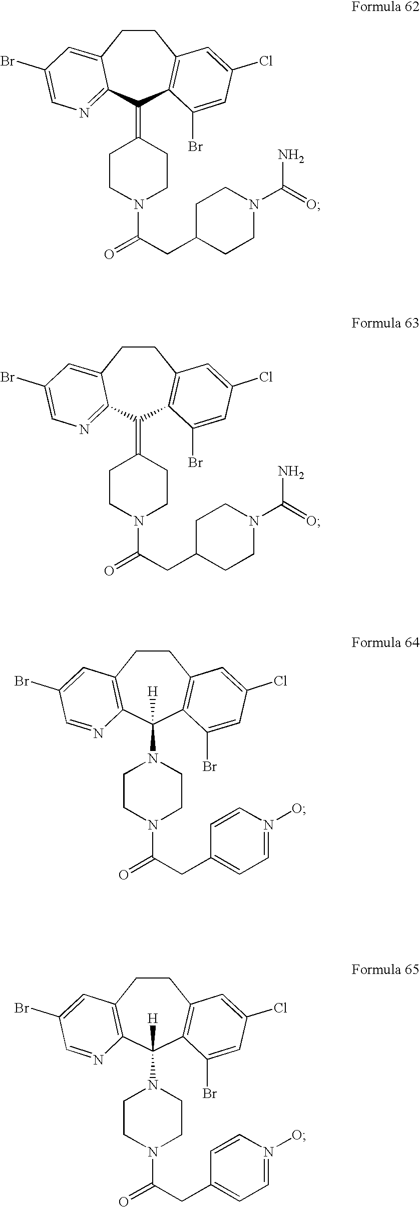

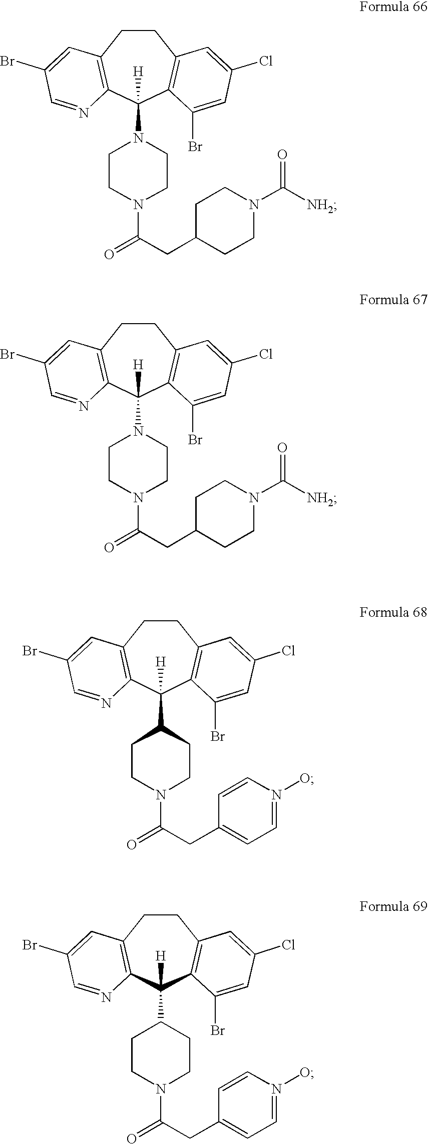

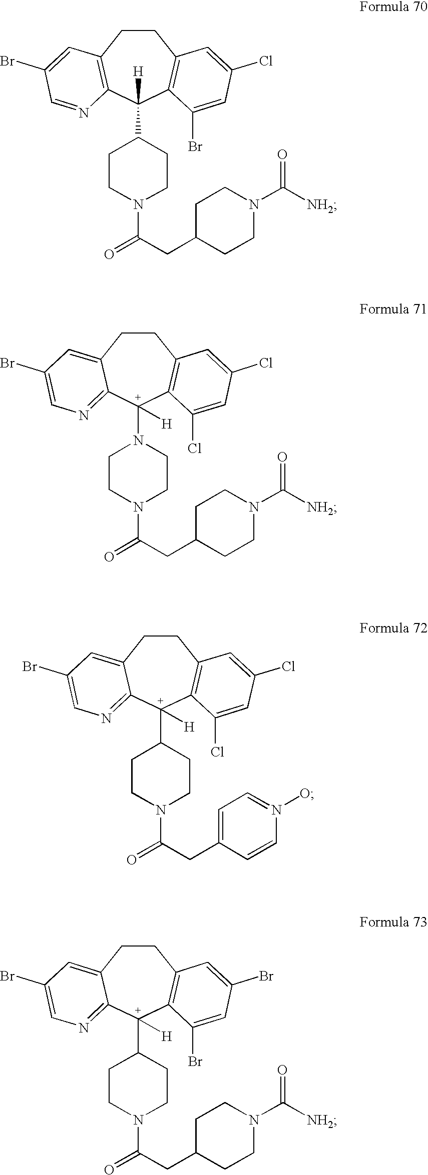

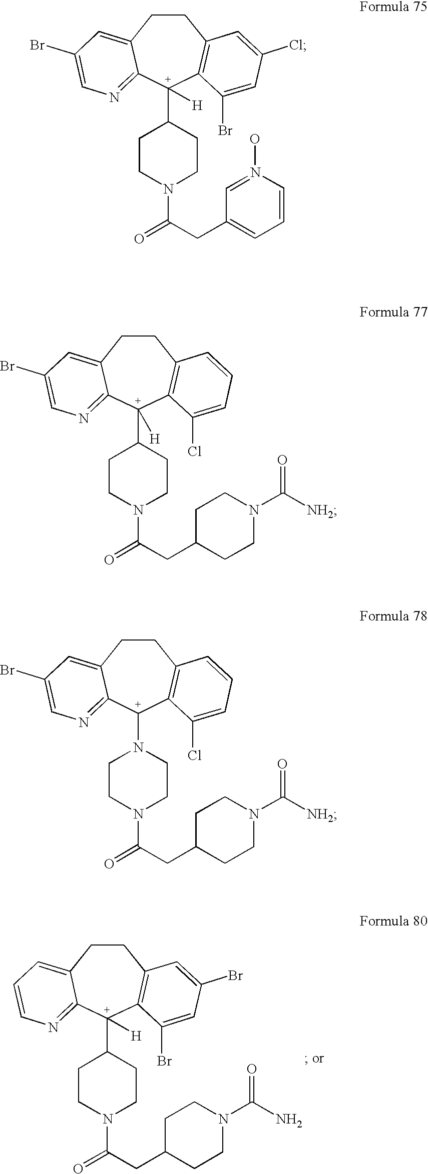

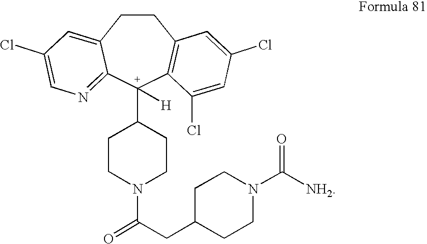

3. A method for treating or preventing a disorder of calcium homeostasis in a subject comprising administering, to the subject, a compound according to the following formula:

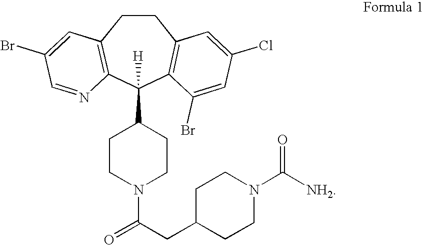

4. The method of claim 1 wherein the medical condition is selected from familial benign hypocalciuric hypercalcemia, neonatal severe primary hyperparathyroidism, renal secondary hyperparathyroidism and osteoporosis.

5. The method of claim 1 wherein the disorder is mediated by a low level of Calcium Ion Sensing Receptor activity or expression or by excess serum parathyroid hormone.

6. The method of claim 1 wherein the subject is a human, a dog or a cat.

7. The method of claim 1 wherein the compound is administered in combination with a pharmaceutically acceptable carrier in a pharmaceutical composition.

8. The method of claim 7 wherein the pharmaceutical composition is in the form of a pill, tablet or capsule.

9. The method of claim 1 wherein the compound of Formula A is administered in association with a second compound for treating a disorder of calcium homeostasis.

10. The method of claim 9 wherein the second compound is selected from AMG073, NPS467, NPS568, gadolinium, lanthanum, neomycin, Mg²⁺, 1,25-dihydroxyvitamin D, calcitrol, paricalcitrol, doxercalciferol, zoledronic acid, calcitonin, alfacalcidol and oxacalcitriol.

11. The method of claim 9 wherein the second compound is administered in combination with a pharmaceutically acceptable carrier in a pharmaceutical composition.

12. The method of claim 11 wherein said compound of Formula A and said second compound are administered in combination with a pharmaceutically acceptable carrier in a single pharmaceutical composition.

13. The method of claim 11 wherein said compound of Formula A and said second compound are administered in combination with pharmaceutically acceptable carriers in two separate pharmaceutical compositions.

14. The method of claim 13 wherein said compound of Formula A and said second compound are administered simultaneously.

15. The method of claim 13 wherein said compound of Formula A and said second compound are administered non-simultaneously.

16. The method of claim 11 wherein the pharmaceutical composition is in the form of a pill, tablet or capsule.

17. A method for decreasing the level of Ca²⁺, parathyroid hormone or parathyroid hormone related protein in the serum of a subject comprising administering a compound according to the following formula to the subject: 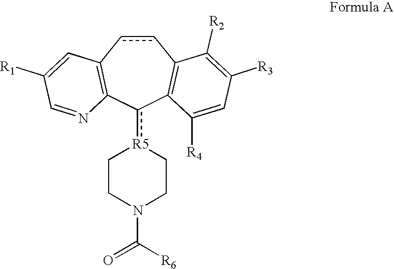
wherein R1 can be hydrogen or halogen
R2, R3 and R4 are independently selected from H and halogen provided that at least one of R2, R3 and R4 is H;
— represents an optional bond;
R5 represents C when the optional bond to R5 is present and represents CH or N when the optional bond to R5 is absent; and
R6 is selected from

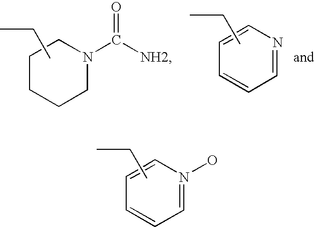

18. A method for treating or preventing a disorder of calcium homeostasis in a subject comprising administering, to the subject, a compound according to the following formula: [chemical structure]


SEQUENCE LISTING (2 sequences)

SEQ ID NO: 1 - protein, 1088 aa, Homo sapiens [sequence omitted]

SEQ ID NO: 2 - protein, 1079 aa, Rattus norvegicus
MASYSCCLALLALAWHSSAYGPDQRAQKKGDIILGGLFPIHFGVAAKDQDLKSRPESVECIRYNFRGFRWLQAMIFAIEEINSSPSLLPNMTLGYRIFDTCNTVSKALEATLSFVAQNKIDSLNLDEFCNCSEHIPSTIAVVGATGSGVSTAVANLLGLFYIPQVSYASSSRLLSNKNQYKSFLRTIPNDEHQATAMADIIEYFRWNWVGTIAADDDYGRPGIEKFREEAEERDICIDFSELISQYSDEEEIQQVVEVIQNSTAKVIVVFSSGPDLEPLIKEIVRRNITGRIWLASEAWASSSLIAMPEYFHVVGGTIGFGLKAGQIPGFREFLQKVHPRKSVHNGFAKEFWEETFNCHLQEGAKGPLPVDTFVRSHEEGGNRLLNSSTAFRPLCTGDENINSVETPYMDYEHLRISYNVYLAVYSIAHALQDIYTCLPGRGLFTNGSCADIKKVEAWQVLKHLRHLNFTNNMGEQVTFDECGDLVGNYSIINWHLSPEDGSIVFKEVGYYNVYAKKGERLFINEEKILWSGFSREVPFSNCSRDCQAGTRKGIIEGEPTCCFECVECPDGEYSGETDASACDKCPDDFWSNENHTSCIAKEIEFLAWTEPFGIALTLFAVLGIFLTAFVLGVFIKFRNTPIVKATNRELSYLLLFSLLCCFSSSLFFIGEPQDWTCRLRQPAFGISFVLCISCILVKTNRVLLVFEAKIPTSFHRKWWGLNLQFLLVFLCTFMQILICIIWLYTAPPSSYRNHELEDEIIFITCHEGSLMALGSLIGYTCLLAAICFFFAFKSRKLPENFNEAKFITFSMLIFFIVWISFIPAYASTYGKFVSAVEVIAILAASFGLLACIFFNKVYIILFKPSRNTIEEVRSSTAAHAFKVAARATLRRPNISRKRSSSLGGSTGSIPSSSISSKSNSEDRFPQPERQKQQQPLSLTQQEQQQQPLTLHPQQQQQPQQPRCKQKVIFGSGTVTFSLSFDEPQKNAMAHRNSMRQNSLEAQRSNDTLGRHQALLPLQCADADSEMTIQETGLQGPMVGDHQPEMESSDEMSPALVMSTSRSFVISGGGSSVTENVLHS